(19) United States
(12) Patent Application Publication
(10) Pub. No.: US 2011/0060225 A1
(43) Pub. Date: Mar. 10, 2011

(54) ULTRASOUND PROBE WITH INTEGRATED PULSERS

(75) Inventors: Scott D. Cogan, Clifton Park, NY (US); Trym Haakon Eggen, Horten (NO); Lukas Bauer, Werder (Havel) (DE); Armin Schoisswohl, Wels (AT); Franz Steinbacher, Pfaffing (AT); Bruno Haider, Ballston Lake, NY (US)

(73) Assignee: General Electric Company, Schenectady, NY (US)

(21) Appl. No.: 12/556,208

(22) Filed: Sep. 9, 2009

(51) Int. Cl. A61B 8/14

(52) U.S. Cl. 600/459

(57) ABSTRACT

Various embodiments of an ultrasound probe for use with an ultrasound system are provided to enable local waveform generation with respect to the ultrasound probe. The ultrasound probe includes a plurality of transducer elements which are independently configured to transmit distinct waveforms. Certain embodiments include a variety of probes that house one or more waveform generators on application specific integrated circuits (ASICs).

### BACKGROUND OF THE INVENTION

The subject matter disclosed herein relates generally to ultrasound imaging, and more particularly, to probes for use in ultrasound imaging.

Medical diagnostic ultrasound is an imaging modality that employs ultrasound waves to probe the acoustic properties of the body of a patient and produce a corresponding image. Generation of sound wave pulses and detection of returning echoes is typically accomplished via a plurality of transducers located in the probe. Such transducers typically include electromechanical elements capable of converting electrical energy into mechanical energy for transmission and mechanical energy back into electrical energy for receiving purposes. Some ultrasound probes include up to thousands of transducers arranged as linear arrays or a 2D matrix of elements.

Since the quality and resolution of a resulting image is largely a function of the number of transducers in such arrays, advanced systems typically incorporate the greatest number of transducers possible. However, since each transducer typically requires a system channel that provides electrical coupling to transmit and receive circuitry and because there are typically a limited number of system channels available, the number of transducers in the probe is effectively limited.

### BRIEF DESCRIPTION OF THE INVENTION

In a first embodiment, a system includes a probe for use with an ultrasound system, the probe comprising a plurality of transducers and a waveform generator configured to transmit a waveform with distinct parameters to each transducer of the plurality of transducers, wherein the waveform generator is located on an application specific integrated circuit (ASIC) located in the probe.

In a second embodiment, a system includes a probe for use with an ultrasound system, the probe comprising a plurality of transducers and a waveform generator coordinated with a system clock and configured to transmit a waveform with distinct parameters to each transducer of the plurality of transducers. The waveform generator includes a bulk delay counter which determines the start time of a digital waveform counter, a waveform counter that counts down to a zero value to determine the number of cycles in the waveform, and a fine delay unit that provides a time delayed waveform to each transducer of the plurality of transducers.

In a third embodiment, a system includes a probe for use with an ultrasound system. The probe comprises a plurality of arrays of transducers, a plurality of pulsers, wherein each transducer of the plurality of transducers receives a signal from a dedicated pulser, wherein each pulser of the plurality of pulsers receives a signal from a dedicated multiplexer, and a plurality of waveform generators configured to transmit a waveform to each transducer of the plurality of transducers, wherein each multiplexer of the plurality of multiplexers is configured to select one waveform from a plurality of waveforms generated by the waveform generator to transmit to its dedicated pulser.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates an embodiment of an exemplary ultrasound data acquisition system including an ultrasound probe and an imaging system in accordance with aspects of the present disclosure;

FIG. 2 illustrates internal components of an exemplary waveform generator including a bulk delay counter, a waveform counter, and a fine delay unit in accordance with aspects of the present disclosure;

FIG. 3 illustrates internal components of an exemplary waveform generator including a run length encoder and a fine delay unit in accordance with aspects of the present disclosure;

FIG. 4 illustrates an embodiment of an exemplary digital 8-bit waveform counter that may be implemented in a waveform generator in accordance with aspects of the present disclosure;

FIG. 5 illustrates exemplary waveform outputs that may be generated in conjunction with a unipolar pulser in accordance with aspects of the present disclosure;

FIG. 6 illustrates exemplary waveform outputs that may be generated in conjunction with a bipolar pulser in accordance with aspects of the present disclosure;

FIG. 7 illustrates an embodiment of an exemplary fine delay unit that may be implemented in a waveform generator in accordance with aspects of the present disclosure;

FIG. 8 illustrates exemplary time delayed waveforms that may be generated during operation of the fine delay unit of FIG. 7 in accordance with aspects of the present disclosure; and

FIG. 9 illustrates an embodiment of an algorithm depicted as steps of exemplary control logic in accordance with aspects of the present disclosure.

### DETAILED DESCRIPTION OF THE INVENTION

As discussed in further detail below, various embodiments of an ultrasound probe communicatively coupled to an imaging system are provided to enable waveform generation proximate to the ultrasound probe. In one embodiment, the ultrasound probe is electronic, reusable, capable of precise waveform timing and intricate waveform shaping for a plurality of independent transducer elements, and capable of communicating analog or digitized data to the imaging system. The disclosed embodiments include a variety of probes that house one or more waveform generators on application specific integrated circuits (ASICs). In some embodiments, such waveform generators may include a bulk delay counter, a waveform counter, and a fine delay unit that cooperate to trigger waveform generation in each of the transducer elements of an array. In other embodiments, the waveform generators may include a run length encoder and a fine delay unit, which cooperate to trigger waveform generation in the transducers while compressing the data required to specify the waveform shape. The foregoing features, among others, may have the effect of achieving precise control over the timing and shape of the waveform transmitted by each transducer while utilizing significantly fewer system channels than the number of transducers. Thus, embodiments of the present disclosure may allow independent control over each transducer without introducing instrumentation complexity to the transmission and waveform generation components of the imaging system. In fact, in such embodiments where the waveform generators are located in the probe, the need for transmit-capable circuitry in the imaging system is potentially eliminated.

In certain embodiments, electronic circuitry integral with the ultrasound probe may include an independent pulser, and multiplexer (MUX) dedicated to each transducer and an independent waveform generator exclusively associated with a sub-array of transducers. In some embodiments, an ASIC may be located opposite the patient-facing surface of the transducer array and similarly dimensioned such that the ASIC surface area is approximately equal to the transducer surface area. In this embodiment, the geometric repetition distance of the transducer elements approximately matches the repetition distance of the pulsers. Additional electronic circuitry included in the systems disclosed herein may include memory, which may be volatile or non-volatile memory (such as read only memory (ROM), random access memory (RAM), magnetic storage memory, optical storage memory, or a combination thereof). Furthermore, a variety of control or operational data may be stored in the memory to provide a specific output (e.g., trigger waveform generation when a clock count reaches zero). As discussed below, certain embodiments of the disclosed ultrasound systems integrate some or all of these features into a probe, which can be readily used to probe a patient and send received signals to the imaging system for further processing.

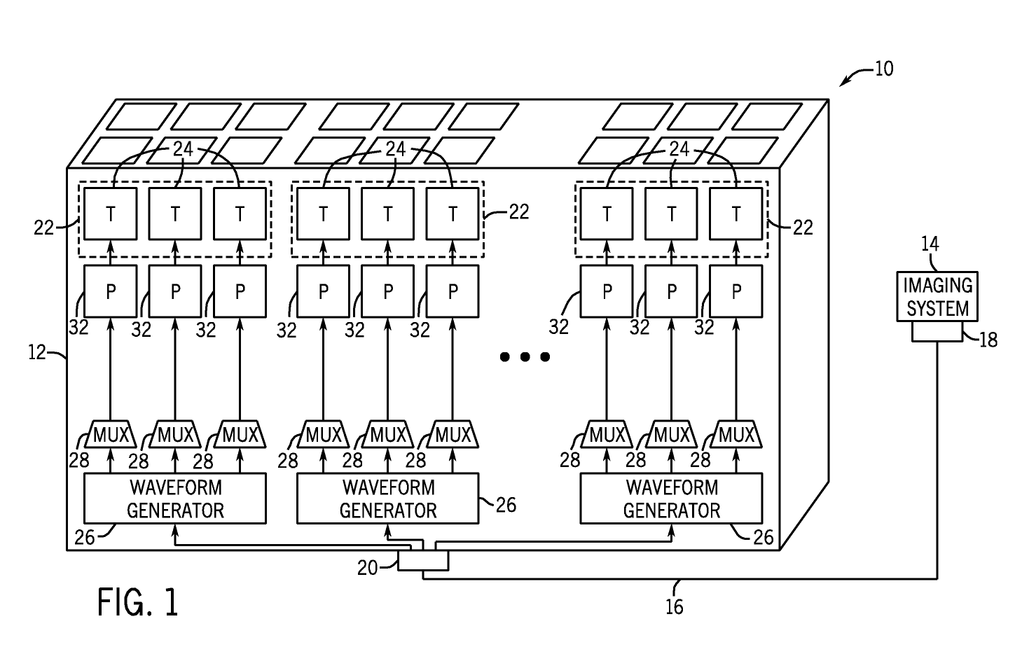

Turning now to the drawings, FIG. 1 schematically depicts an exemplary ultrasound data acquisition system 10 that includes an ultrasound probe 12 (hereinafter, “the probe”) and an imaging system 14 in accordance with aspects of the present disclosure. In some embodiments, the probe 12 may include a handle portion (e.g., a grooved section designed for gripping) configured to facilitate use by an operator, such as a medical technician. Additionally, it should be noted that the probe 12 may be manufactured to take on any of a number of geometries, such as a t-shape, a rectangle, a cylinder, and so forth. In certain embodiments, the imaging system 14 may include keyboards, data acquisition and processing controls, an image display panel, user interfaces, and so forth. As illustrated, the probe 12 and the imaging system 14 may be communicatively coupled via a data conduit 16, which connects a first terminal 18 integral with the imaging system 14 to a second terminal 20 integral with the probe 12. It should be noted that the data conduit 16 may transmit digital or analog data between the imaging system 14 and the probe 12.

The data conduit 16 may facilitate the bidirectional exchange of data between the probe 12 and the imaging system 14. For instance, in some embodiments, the imaging system transmits control signals (e.g., counter start values) to the probe 12 and receives matrices of digital data or analog signals representing reflection signals returned from tissue interfaces within the patient during a pulse-echo data acquisition method. Additionally, in some embodiments, the imaging system 14 may include a main controller, and the probe 12 may include a secondary controller. The main controller and the secondary controller may communicate analog or digital data via the bidirectional conduit 16 during operation. For instance, the main controller may send the secondary controller a first set of signals via the bidirectional conduit 16, wherein the number of signals in the first set is limited by the number of data cables that can practically be routed between the imaging system 14 and the probe 12. The secondary controller located in the probe may contain circuitry configured to expand the set of signals sent by the main controller into a secondary set of signals for use in the probe 12 that is much larger and/or more complete than the first set of signals. Accordingly, the secondary controller may contain memory, which may be volatile or non-volatile memory, such as read only memory (ROM), random access memory (RAM), magnetic storage memory, optical storage memory, and so forth, for storing and/or processing the signals.

The probe 12 includes sub-arrays 22 of transducers 24 that are configured to produce and detect ultrasound waves. Each individual transducer 24 is generally capable of converting electrical energy into mechanical energy for transmission and mechanical energy into electrical energy for receiving purposes. In certain embodiments, the transducers 24 may be voltage biased when receiving echoes back from the patient. That is, the transducers 24 may be precharged to a certain voltage (e.g., 1v, 2v) prior to receiving signals back from the patient such that all received signals take on a positive value. The foregoing feature may have the effect of simplifying electrical circuitry associated with the receiving cycle in certain embodiments. In some embodiments, each transducer 24 may include a piezoelectric ceramic, a matching layer, an acoustic absorber, and so forth. Additionally, the transducers 24 may be of any type suitable for use with diagnostic ultrasound, such as broad-bandwidth transducers, resonance transducers, and so forth. In the illustrated embodiment, the transducer array is comprised of multiple sub-arrays 22. The sub-array 22 is depicted as a 3×2 matrix of transducers 24. However, it should be noted that in other embodiments, more or fewer transducers 24 may be included in each sub-array 22. For instance, in one embodiment, the sub-array 22 may include twenty five transducers 24 arranged in a 5×5 matrix. In other embodiments, the sub-array 22 may be a 2×2 matrix, a 4×4 matrix, a 10×10 matrix, or any other matrix size suitable for diagnostic imaging. In other embodiments, the sub-array 22 may be of non-rectangular shape, for example, triangular, hexagonal, octagonal or an other suitable shape. Additionally, it should be noted that the geometric repetition distance of the transducer elements approximately matches the repetition distance of the pulsers.

In the depicted embodiment, one waveform generator 26 is dedicated to each sub-array 22 of transducers 24. That is, in the illustrated embodiment, each waveform generator 26 is configured to generate signals for the six transducers 24 of the associated sub-array 22, e.g., the respective 3×2 matrix of transducers 24. In some embodiments, each waveform generator 26 may be located on an ASIC in the probe 12. For example, each ASIC may be located opposite the tissue facing surfaces with respect to the transducers 24 and may be dimensioned such that the ASIC surface area is approximately equal to the transducer surface area. In such an embodiment, the electrical circuitry associated with achieving waveform generation and beamforming may be located on a small ASIC area, reducing complexity and monetary cost of the ultrasound data acquisition system 10 as compared to traditional designs. In further embodiments, pulser 32 circuitry may be located on a high voltage ASIC while remaining digital circuitry may be located on a separate stacked ASIC that is encapsulated in a single package with the high voltage ASIC. The foregoing features, among others, may have the effect of facilitating the use of the ultrasound data acquisition system 10 disclosed herein with Doppler ultrasound imaging.

In the depicted embodiment, the waveform generators 26 output signals to multiplexers 28, which are each dedicated to the control of an individual transducer 24. Accordingly, each MUX 28 selects a waveform from a plurality of waveforms received from the waveform generator 26 to send to the transducer 24 it controls. For instance, if the waveform generator 26 outputs eight waveforms of various delays, the MUX 28 size will be 8:1, and the MUX 28 will select one waveform out of eight possibilities to send to its associated transducer 24. In certain embodiments, the size of the multiplexers 28 is determined by properties of internal components of the waveform generators 26, as discussed in more detail below.

In some embodiments, each transducer 24 may be associated with a respective pulser which receives a signal from the MUX 28. For instance, a respective pulser 32 may receive control signals at a low voltage (e.g., 3.3V or 5.0V) and produce high voltage (e.g., negative 100V to positive 100V) signals that drive the transducer elements 24. The low voltage control signal may be a digitally encoded representation of the desired pulser state. Additionally, the pulser 32 having such functionality may receive a timing signal of a preset number of bits and generate a variety of independent signals from the information encoded in the received bits. For example, a timing signal of two bits which may be decoded to generate four independent signals for four pulser states (e.g., high, low, ground, receive). It should be noted that any number of suitable bits may be encoded as the timing signal and any number of possible signals may be generated based on the number of received bits.

The pulsers 32 function as transmitters, which provide the voltage needed to excite the piezoelectric material (e.g., a ceramic) in the transducers 24. Accordingly, the pulsers 32 control the power transmitted to the patient via adjustment of an applied voltage. It should be noted that in some embodiments, a digital-to-analog converter may cooperate with the pulsers 32 or other elements contained in the probe 12 to determine the amplitude of the applied voltage. In some embodiments, such as in a pulse echo operation mode, the pulsers 32 may pulse their respective transducers 24 at frequencies of several Megahertz. Since each transducer 24 in the probe 12 has a dedicated pulser 32 and MUX, and each sub-array 22 of transducers 24 has a dedicated waveform generator 26 located within the probe 12, each transducer 24 may be individually controlled without the introduction of instrumentation complexity or system noise.

In some embodiments, the fine delay unit 38 may be comprised of multiple delay stages with independent Muxes. For example, in the first fine delay stage the Mux complexity may be reduced by accessing only a subset of the delay stages (e.g. every other). A second fine delay stage may create two delayed versions of the control signal that are separated by a single delay step. In some embodiments, this may reduce hardware complexity compared to an implementation with a single Mux with more inputs.

In some embodiments, the pulsers 32 may be configured to transition into a receive state and communicate with receiver circuitry located in the probe 12. For instance, the receiver circuitry may include a low noise amplifier (LNA), which is configured to amplify weak signals received from the patient, thus ensuring that information contained in signals of low strength is not lost. In further embodiments, the LNA may output the amplified signal to a modulator with a selectable mixer clock phase, which selects an operating phase by shifting the frequency of the received signals. For instance, if a signal is received with a frequency of 5 MHz, and the clock frequency is 20 MHz, the modulator may generate a 25 MHz signal. In this way, the modulator may change the phase of the 20 MHz clock from channel to channel.

The receiver circuitry may also include a time gain compensation amplifier (TGCA) that changes the gain of the received signal over time during operation. Such a TGCA may be necessary since echo signals from areas of the patient located in close proximity to the probe may require different levels of amplification than echo signals from patient areas located further from the probe. A delay circuit may also be included in the receiver circuitry to correct for signals that may be received at a variety of angles from many different areas of the patient. In some embodiments, dedicated receiver circuitry may be associated with each pulser 32 in the probe 12. In such embodiments, summation circuitry located in the probe 12 may combine the inputs from the components of the receiver circuitry associated with multiple transducers 24 prior to the transmission of the received data to the imaging system 14.

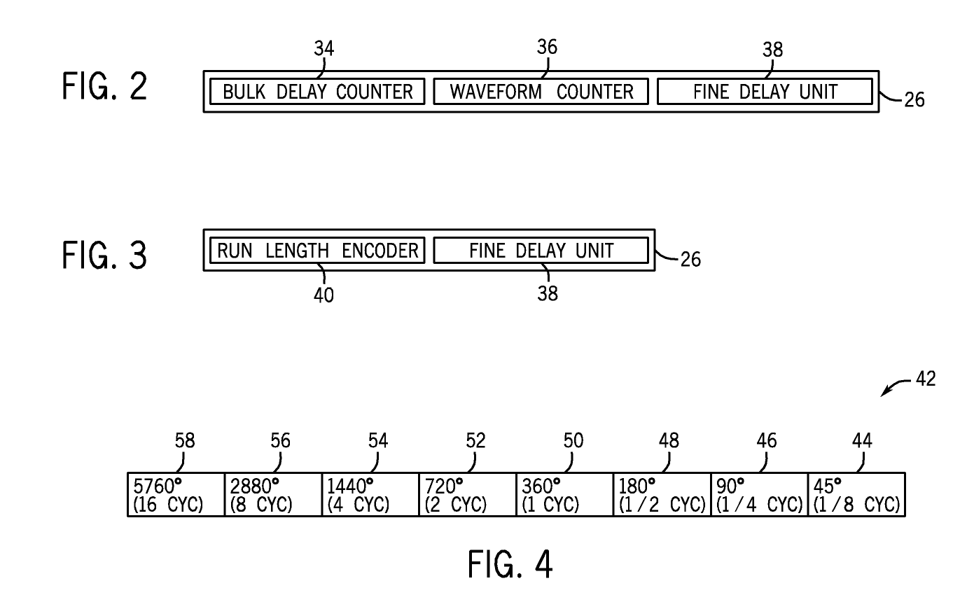

FIGS. 2 and 3 illustrate exemplary internal components of one embodiment of a waveform generator 26, which include a bulk delay counter 34, a waveform counter 36, and a fine delay unit 38. In the embodiment illustrated in FIG. 2, the bulk delay counters 34 in the different respective waveform generators 26 are triggered to start at the same instance. However, each bulk delay counter 34 in each waveform generator 26 begins at a different start value. When each bulk delay counter 34 has decremented down to zero, its respective waveform counter 36 begins counting to determine the length of the waveform which will be transmitted to the fine delay unit 38. That is, the waveform counter 36 is configured to determine how many times the waveform will oscillate, whether there will be a short burst or a long burst, and so forth. Since each bulk delay counter 34 may begin at a different start value, each waveform counter 36 can be triggered at a different time point. The output of the waveform counter 36 becomes the input for the fine delay unit 38, which generates a plurality of delay versions of the same waveform that may then be transmitted to individual transducers 24 in a given sub-array 22, through the MUX. For instance, in one embodiment, the fine delay unit 38 may output eight delayed versions of the waveform to the sub-array 22 of transducers 24. Each transducer element has a MUX which may then select a respective delay waveform for each of the transducers 24 in the sub-array 22.

FIG. 3 illustrates a further embodiment of the waveform generator 26 illustrated in FIG. 2. In this embodiment, a run length encoder (RLE) 40 may replace the functionalities of the bulk delay counter 34 and the waveform counter 36. That is, the run length encoder 40 may provide an efficient means of describing complex waveforms using a small amount of configuration data.

FIG. 4 illustrates an exemplary digital 8-bit waveform counter 42 that may be located in the waveform generator 26. The counter may be set to an initial value and count down to zero upon a trigger from the bulk delay counter. The depicted embodiment of the counter 42 includes three bits 44, 46, and 48, which cooperate to define a width of a first pulse in the encoded waveform. Each bit oscillates at half the frequency of the bit to its right. For instance, during operation, the least significant bit 44 oscillates between zero and one on each clock cycle. The next bit 46 oscillates at half the frequency of bit 44 (i.e., ¼ cycle instead of ⅛ cycle). Similarly, the next bit 48 oscillates at half the frequency of bit 46 (i.e., ½ cycle instead of ¼ cycle). In one embodiment, bit 48 is configured to oscillate a preset number of times based on an input start value. In this way, the oscillations of the three bits 44, 46, and 48 between zero and one define the width of the first pulse of the waveform.

In one embodiment, the remaining bits 50, 52, 54, 56, 58 are used during operation to implement the desired number of cycles. For instance, in the illustrated embodiment, the most significant bit 58 may be used to encode sixteen cycles, which means the resulting waveform may have up to sixteen pulses. The entire 8-bit counter 42, therefore, is capable of configuring 256 different waveforms with various pulse widths and number of pulses. The waveform generation can be considered completed when the counter reaches zero, or possibly some other stop value. If the other stop value is non-zero, this will have the effect of limiting the final pulse width.

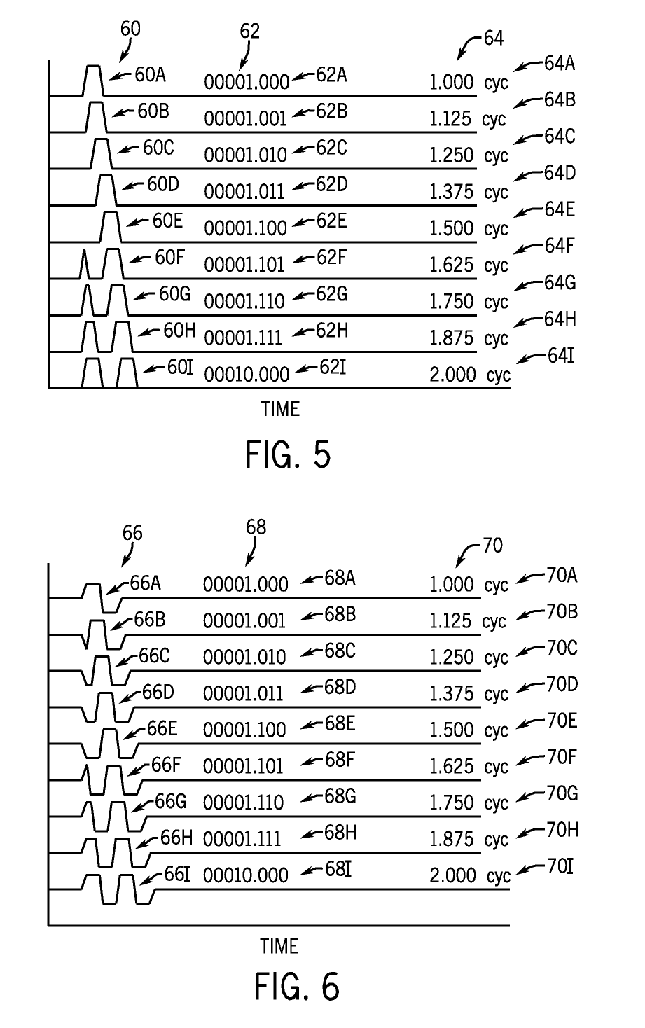

FIGS. 5 and 6 illustrate how the exemplary 8-bit counter 42 illustrated in FIG. 4 may operate to generate waveforms. FIG. 5 illustrates possible waveform outputs 60 that may be generated in conjunction with a unipolar pulser when exemplary initial values 62 are input into an exemplary 8-bit waveform counter 42 to produce various transmit waveform cycle lengths 64. The center frequency of the waveforms 60 are determined in large part by the clock frequency which runs the waveform counter 42. In one embodiment, the unipolar pulser transistors are configured to pull to one high value (e.g., +60 volts) and one low value (e.g., 0 volts) during operation and may return back to zero volts after operation commences. However, it should be noted that pull low transistors or bipolar transistors, as discussed in more detail below, may also be used in conjunction with the 8-bit counter 42.

The first exemplary waveform 60A that is generated from initial value 62A is a single pulse of a defined width. In this example, the resulting waveform is driven by the value of the third bit 48 in the waveform counter 42 as the counter counts down to zero. As the cycle count 64A increases by ⅛ to cycle 64B, the initial value of the least significant bit 44 of the 8-bit counter 42 has been changed from zero to one and the pulse generated in waveform 60B has the same width as waveform 60A but a later starting time. Similarly, as the cycle count 64B increases by ⅛ to cycle count 64C, the least significant bit 44 of the 8-bit counter 42 has been changed from one to zero, the next bit 46 has been changed from zero to one, and the pulse generated in waveform 60C has the same width as waveforms 60B and 60A but at a later starting time. The same pattern continues for waveforms 60D and 60E with initial values 62D and 62E and cycle counts 64D and 64E, in which each successive waveform shown starts at a delayed time with respect to the previous waveform due to the difference in the initial values. At initial value 62F, the waveform 60F that is generated begins to reflect another waveform peak. The width of the new waveform peak is increased in waveform 60G as compared to waveform 60F as the initial value changes from 62F to 62G and the cycle count changes from 64F to 64G. The same pattern continues for waveforms 60H and 60I with initial values 62H and 62I and cycle counts 64H and 64I, where each successive waveform is extended in length by ⅛ cycle. The waveform 60I corresponds to the initial value 62I, which leads to the generation of two peaks of equal width. In this way, the initial values 62 dictate the waveforms 60 pulse width and number of pulses generated by the unipolar pulser.

FIG. 6 illustrates exemplary waveform outputs 66 that may be generated in conjunction with a bipolar pulser when exemplary initial values 68 are input into the 8-bit waveform counter 42. In one embodiment, as illustrated, a bipolar transistor is configured to pull to one high value (e.g., +60 volts) and one low value (e.g., −60 volts) during operation and may pull back to zero volts after operation commences. The first exemplary waveform 66A that is generated from initial value 68A during cycle count 70A consists of a first upward pulse of a defined width and a first downward pulse of an equal width. As cycle count 70A increases by ⅛ to cycle count 70B, the least significant bit 44 of the 8-bit counter 42 has been changed from zero to one and the length of the generated waveform 66B has increased by ⅛ cycle, with an additional short downward peak at the beginning of the pulse in waveform 66B as compared to waveform 66A. Similarly, as cycle count 70B increases by ⅛ to cycle count 70C, the least significant bit 44 of the 8-bit counter 42 has been changed from one to zero, the next bit 46 has been changed from zero to one, and the initial downward pulse in waveform 66C is widened as compared to waveform 66B.

The same pattern as described above continues for waveforms 66D and 66E with initial values 68D and 68E and cycle counts 70D and 70E, where the initial downward peak widens to its ultimate width in waveform 66E. At initial value 68F, the waveform 66F begins to reflect an additional initial upward peak. The width of this additional upward peak can be incrementally increased by using initial values 68F, 68G, 68H and 68I such that a full two cycle waveform is generated in waveform 66I. In this way, the initial values 68 dictate the waveforms 68 pulse width and number upward and downward pulses generated by the bipolar transistor. In other words, the initial values 68 determine the bandwidth and center frequency of the resulting waveforms 68.

In the embodiments illustrated herein, the pulse width of the first half cycle of the generated waveform may be determined by the counters, and the waveform subsequently oscillates at a predetermined frequency. However, it should be noted that in other embodiments, pulser elements with greater sophistication may be used in conjunction with aspects of the present disclosure to exhibit greater control over the generated waveform. For instance, in one embodiment, a pulser capable of pulling to five voltage levels (e.g., −60v, −30v, 0v, 30v, 60v) may be used to exhibit greater control over the shape of the generated waveform. In such an embodiment, both the width of each pulse and the length of time the waveform is maintained at a particular voltage may be controlled. This embodiment may give rise to the need to include a supplemental counter that may be located between the waveform counter 36 and the fine delay unit 38 in the embodiment illustrated in FIG. 2. In certain embodiments, the supplemental counter may store the width of each half cycle separately and may digitally encode how long each of the five voltage levels will be maintained. This would be considered a run length encoded waveform generation scheme.

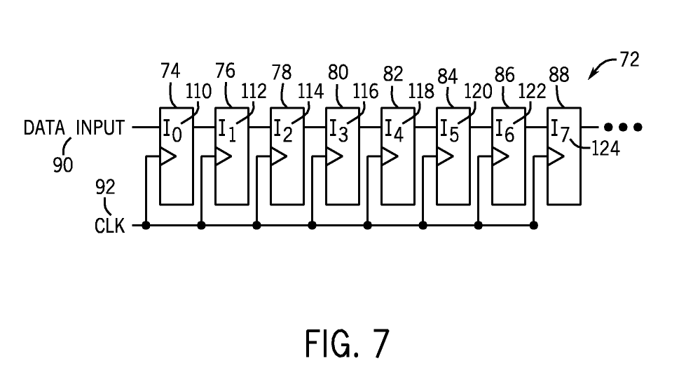

FIG. 7 illustrates an exemplary fine delay unit 72 that may be located in the waveform generator 26 and may receive its input from the waveform counter 36, i.e. from the ½ cycle bit 48. In the depicted embodiment, the illustrated fine delay unit 72 is an 8-bit chain that functions as a serial in serial out shift register with eight flip-flops 74, 76, 78, 80, 82, 84, 86, 88. However, it should be noted that in alternative embodiments, more or fewer delay stages may be used in the chain, or an alternative delay method, such as a chain of capacitors, may be used. An encoded waveform signal is fed into data input 90 to the fine delay unit 72. A clock signal 92, which is fed in parallel to the flip-flops 74, 76, 78, 80, 82, 84, 86, 88, triggers the input data signal to be shifted from an adjacent flip-flop to the next flip-flop. In this way, the flip-flop outputs 110, 112, 114, 116, 118, 120, 122 represent time-delayed versions of the input data signal.

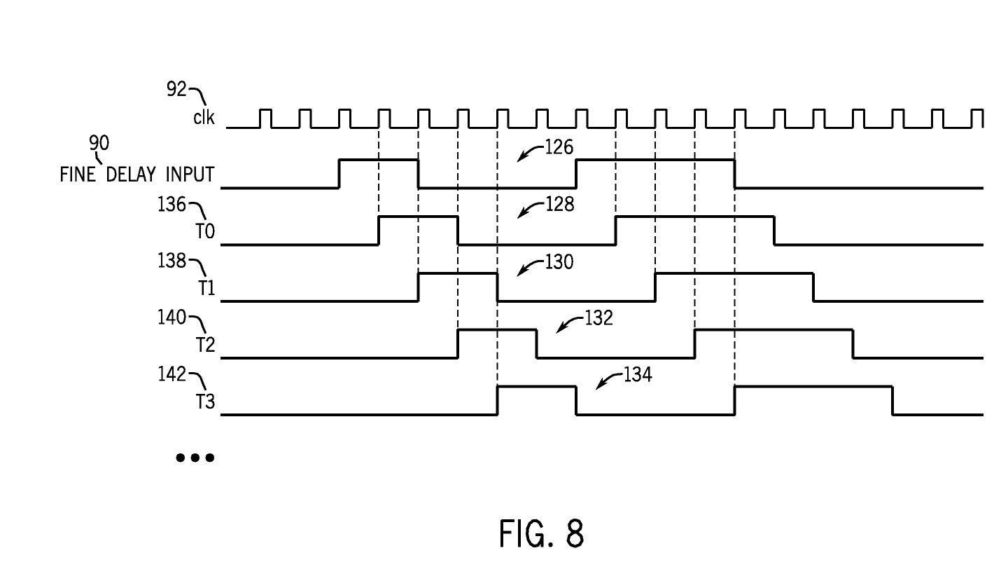

FIG. 8 illustrates how the flip-flop chain 72 of FIG. 7 may be used to generate a plurality of time delayed waveforms that may be transmitted by the pulser 32 during operation. In the illustrated embodiment, the fine delay input 90 is represented as the digital sequence 00110000111100, which is also shown as waveform 126. As the digital sequence, 00110000111100, is transmitted sequentially through the flip-flop chain 72, time-delayed versions of the waveform 128, 130, 132, 134 are generated by the fine delay unit and made available to the transducer MUXs. For instance, T0, as indicated by reference numeral 136, corresponds to the waveform made available from the flip-flop output 110 in FIG. 7. Similarly, T1, as indicated by reference numeral 138, corresponds to the waveform made available from the flip-flop output 112 in FIG. 7. Accordingly, T2, as represented by reference numeral 140, and T3, as represented by reference numeral 142, correspond to the waveforms made available from the flip-flop outputs 114 and 116, respectively. It should be noted that in other embodiments, more or fewer transistors may be in the sub-array and longer or shorter fine delay inputs may be transmitted through the flip-flop chain 72.

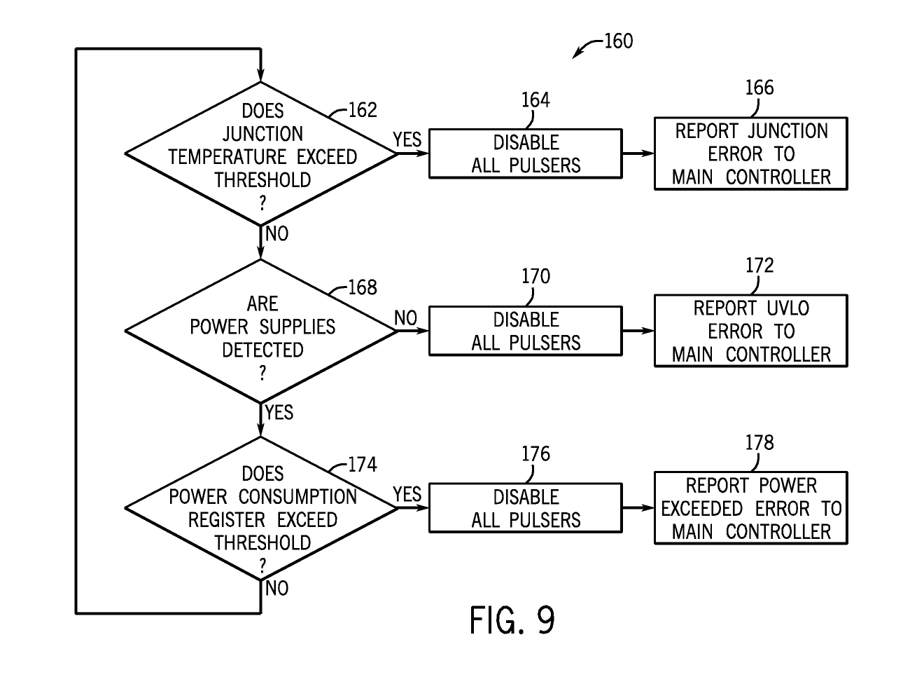

While the preceding relates various aspects of waveform generation as may be implemented in the probe 12, such as by one or more ASICs provided in the probe 12, other features may also be present as part of the probe 12 in certain embodiments. For example, FIG. 9 illustrates control logic 160 that may be provided as a computer-implemented algorithm or as a hardware control loop to ensure thermal protection of the electronics located in the ultrasound probe 12 during use. In one such embodiment of the control logic 160, a controller may check to see if a junction temperature in the ASIC exceeds a preset threshold, as represented by block 162. For instance, in some embodiments, circuitry on the ASIC may monitor junction temperatures to avoid overheating of the probe 12 or the ASIC itself. In one embodiment, the threshold value may be set approximately between 105° C. and 125° C. or at any other suitable value. If the controller detects that the junction temperature exceeds this threshold, the pulsers 32 in the probe 12 are disabled, as represented by block 164, and a junction error is reported to a main system controller, as represented by block 166. If the junction temperature is below the threshold, the controller may then check to ensure that all the system power supplies are detected, as represented by block 168. This under-voltage lock-out (UVLO) check may prevent the possibility that any missing power supply, which could result in erroneous operation or damage to the electronics, is identified as missing before system damage occurs. If a power supply is missing, the pulsers 32 in the probe 12 are disabled, as represented by block 170, and a UVLO error is reported to the main controller, as represented by block 172.

If all the power supplies are detected, the controller checks whether an estimated power consumption register has exceeded a threshold value, as represented by block 174. For instance, in one embodiment, the estimated power consumption register may be a digital counter that is incremented by an amount that correlates with estimated or measured parameters indicative of the thermal state of the transducer at certain time intervals (e.g., every 40 ns). For example, the estimated power consumption register may be incremented by an amount that corresponds to an estimate of the power dissipated by the pulsers, which may correspond to a supply voltage or by an amount that corresponds to a measured value of the actual supply voltage. Additionally, the estimated power consumption register is periodically drained to simulate the natural thermal dissipation over time (e.g., a certain value may be subtracted at preset time intervals). In this way, the register represents an estimated instantaneous temperature level or thermal state. If the estimated power consumption register exceeds a threshold value, the pulsers 32 in the probe 12 are disabled, as represented by block 176, and a power exceeded error is reported to the main controller, as represented by block 178. The controller may cycle through blocks 162, 168, and 174 throughout operation, continually ensuring that thermal damage does not occur to the electronics located in the probe 12.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An ultrasound probe, comprising:
a plurality of transducers; and
one or more application specific integrated circuits (ASICs) configured to generate and transmit waveforms with distinct parameters to each transducer of the plurality of transducers.

2. The ultrasound probe of claim 1, wherein each ASIC is located opposite the patient facing surfaces of the plurality of transducers and wherein each ASIC is dimensioned such that an ASIC surface area is approximately equal to a surface area of a transducer array.

3. The ultrasound probe of claim 1, wherein the distinct parameters comprise one or more of waveform shape or waveform timing delay.

4. The ultrasound probe of claim 1, comprising a plurality of pulsers, wherein each transducer of the plurality of transducers receives a signal from a dedicated pulser of the plurality of pulsers.

5. The ultrasound probe of claim 4, wherein the geometric repetition distance between each adjacent transducer of the plurality of transducers is approximately equal to the repetition distance between each adjacent pulser of the plurality of pulsers.

6. The ultrasound probe of claim 4, comprising a plurality of multiplexers, wherein each pulser of the plurality of pulsers receives a signal from a dedicated multiplexer of the plurality of multiplexers.

7. The ultrasound probe of claim 6, wherein each multiplexer of the plurality of multiplexers is configured to select one waveform from a plurality of waveforms generated by the waveform generator to transmit to its dedicated pulser.

8. The ultrasound probe of claim 1, wherein each waveform generator comprises a digital bulk delay counter configured to determine a start of a digital waveform counter.

9. The ultrasound probe of claim 1, wherein each ASIC comprises a digital waveform counter configured to determine a number of cycles of the waveform.

10. The ultrasound probe of claim 1, wherein each ASIC comprises a fine delay unit configured to generate a plurality of delay versions of the waveform.

11. An ultrasound probe, comprising:
a plurality of transducers; and
a waveform generator coordinated with a system clock and configured to transmit a waveform with distinct parameters to each transducer of the plurality of transducers, the waveform generator comprising:
a digital waveform counter that determines the number of cycles in the waveform;
a bulk delay counter which determines the start of the digital waveform counter; and
a fine delay unit that provides a time delayed waveform to each transducer of the plurality of transducers.

12. The ultrasound probe of claim 11, wherein the bulk delay counter and the waveform counter comprise a run length encoder.

13. The ultrasound probe of claim 11, wherein the waveform counter is configured to generate a digital output that drives pull-up transistors, pull-down transistors, or a combination thereof.

14. The ultrasound probe of claim 11, wherein the waveform generator comprises an application specific integrated circuit.

15. The ultrasound probe of claim 11, comprising a plurality of pulsers, wherein each transducer of the plurality of transducers receives a signal from a dedicated pulser of the plurality of pulsers.

16. The ultrasound probe of claim 15, comprising a controller configured to disable the plurality of pulsers when thermal indicators exceed a preset threshold.

17. A system, comprising:
a probe for use with an ultrasound system, the probe comprising:
a plurality of sub-arrays of transducers;
a respective waveform generator for each sub-array of transducers, wherein each waveform generator is configured to generate a plurality of delay differentiated waveforms;
a plurality of multiplexers associated with each respective waveform generator, is configured such that each multiplexer of the plurality of multiplexers selects one waveform of the plurality of delay-differentiated waveforms generated by the respective waveform generator and to transmit the selected waveform to a dedicated transducer of the sub-array of transducers associated with the respective waveform generator; and
an imaging system communicatively coupled to the probe via a bidirectional conduit.

18. The system of claim 17 comprising, a plurality of pulsers, wherein each pulser is associated with a respective transducer, each pulser configured to transmit the selected waveform to the respective transducer.

19. The system of claim 17, wherein the waveform generator comprises a digital bulk delay counter configured to determine the start of a digital waveform counter.

20. The system of claim 17, wherein the waveform generator comprises a digital waveform counter configured to determine a number of cycles of a generated waveform.
